(19) United States
(12) Patent Application Publication
(10) Pub. No.: US 2018/0028336 A1
(43) Pub. Date: Feb. 1, 2018

(54) SUPPORT APPARATUS WITH ADJUSTABLE STIFFNESS

(71) Applicant: PEACOCKS ORTHOTICS LIMITED, New Castle upon Tyne, Tyne, and Wear (GB)

(72) Inventors: Jari Pallari, Newcastle upon Tyne, Tyne, and Wear (GB); Stephen James Cook, Newcastle upon Tyne, Tyne, and Wear (GB)

(21) Appl. No.: 15/551,151

(22) PCT Filed: Feb. 11, 2016
(86) PCT No.: PCT/EP2016/052928

§ 371 Date: Aug. 15, 2017

(30) Foreign Application Priority Data
Feb. 19, 2015 (EP) 15155760.0
Feb. 19, 2015 (GB) 1502798.0

(51) Int. Cl. A61F 2/66; A61F 5/14; A61F 5/01; A43B 7/14

(52) U.S. Cl. CPC A61F 2/66; A43B 7/141; A61F 5/14; A61F 5/0111; A61F 2002/503

(57) ABSTRACT

A foot orthotic having a body adapted to support a foot of a user is disclosed. The foot orthotic has a stiffness adjusting member (6) adapted to be mounted to the body to adjust the stiffness of the orthotic. Protrusions on the body enable the stiffness adjusting member (6) to be mounted to the body.

This invention relates to a support apparatus having adjustable stiffness for providing tailored support to a body part of a user, and relates particularly, but not exclusively, to an orthotic and/or prosthetic apparatus.

Orthotic devices, or orthoses, are medical devices used to treat or prevent a number of patient pathologies or musculoskeletal problems, or to enhance a user's performance. For example, conditions such as plantar fasciitis, pressure lesions on the feet, pain from arthritis, and conditions in other parts of the body that require the modification of kinetics in the lower limbs and feet, can be treated with foot orthoses.

A simple foot orthotic comprises a form-fitting insole that usually conforms to the shape of a user's foot on the upper side and can have a generic shoe-fitting shape on the lower side. In some cases, the foot orthotic is a shell of largely uniform thickness with a heel element added.

Foot orthotics work by altering the kinematics of the forces experienced by the user's foot in a desired way and can consequently reduce forces acting in another part of the body during the gait cycle. They can also be used to redistribute the pressure on the foot to reduce pathological forces or alter the centre of mass of the user.

Foot orthotics utilise a range of materials to treat patient pathologies. These materials include carbon fibre, ethylene-vinyl acetate, gels, and polypropylene. Most foot orthotics are mass-produced but bespoke foot orthotics are also made to treat a patient in a more focussed and effective way.

3D printing, or additive manufacturing, methods involve laying down successive layers of material to create a three-dimensional object according to the information in a digital file, such as a CAD file. In this way, appropriate modelling software can accept a file containing information from a three-dimensional scan as input, allow a user to virtually manipulate the scanned object, and subsequently print the object as a new three-dimensional body.

An example of the use of additive manufacturing is the creation of foot orthotics. A three-dimensional scan of a user's foot is uploaded to a computer and a negative cast of the foot sole is designed in software. In combination with data such as the person's gait, or medical or athletic requirements, this negative cast is shaped as required into a bespoke foot orthotic device able to be printed by a 3D printer.

In order to meet the needs of the user of an orthotic device, the device must respond in a desired mechanical way to the user's motion. To do this, the device is required to have a particular shape and stiffness. A device which is not bespoke will not be shaped specifically to a body part of the user and as such cannot provide a tailored mechanical response to the user. If an orthotic device has a largely uniform thickness, its stiffness will also be largely uniform, meaning the device will be unable to provide areas of localised support to the body part of the user.

The needs of the user of an orthotic device change with time. As the user's body changes, such as in response to use of an orthotic device, the user may require a new orthotic device with a different shape or different stiffness specifications to best serve his or her needs after a period of time. This would require being measured for and purchasing another device.

Additive manufacturing methods can also be used to create prosthetic apparatus, for example prosthetic sockets such as transtibial/femoral/humeral/radial prosthetic sockets. Prosthetics replace a part of the anatomy, unlike orthoses which support an existing part. Generally speaking the interface between a prosthetic limb and the residual limb is the most difficult part of the prosthetic to make and these are always bespoke as every residual limb is a different shape. This significantly increases the cost and difficulty of manufacture of the prosthetic apparatus.

Preferred embodiments of the present invention seek to overcome one or more of the above disadvantages associated with the prior art.

According to a first aspect of the present invention, there is provided a support apparatus comprising a body adapted to support a body part of a user, and at least one stiffness adjusting member adapted to be mounted to said body to adjust the stiffness of the apparatus, wherein said body comprises first engaging means and at least one said stiffness adjusting member comprises respective second engaging means for engaging said first engaging means to mount the stiffness adjusting member to the body.

By providing at least one stiffness adjusting member adapted to be mounted to said body to adjust the stiffness of the apparatus, wherein said body comprises first engaging means and at least one said stiffness adjusting member comprises respective second engaging means for engaging said first engaging means to mount the stiffness adjusting member to the body, this provides the advantage of making the mechanical properties of the apparatus more adjustable.

The apparatus can also have its stiffness altered in more than one location on the device at any one time, providing the advantage of further increased adjustability of the apparatus.

The apparatus can have its stiffness modified repeatedly by means of re-engaging at least one said stiffness adjusting member to another location on the body at any time, providing the advantage of increased adaptability of the apparatus to the user's changing needs over time.

Said first and/or second engaging means may comprise at least one respective protrusion.

Said first and/or second engaging means may comprise at least one respective recess.

Said first and/or second engaging means may comprise a respective plurality of said recesses and/or protrusions or least one said recess and one said protrusion.

These features provide the advantage of enabling a stiffness adjusting member to be placed at a plurality of locations on the body.

Said body and/or said first engaging means and/or said second engaging means and/or at least one said stiffness adjusting member may be manufactured by additive manufacturing.

This provides the advantage that the body and first engaging means form a unitary body that has greater inherent robustness than a similar body and similar engaging means having been coupled together with an adhesive or other fixing means. It also provides the advantage that the shape of the apparatus and/or the shapes of its components are more accurately reproduced with respect to the user's requirements.

The apparatus may be an orthotic and/or prosthetic apparatus.

According to a second aspect of the present invention, there is provided a method of altering the stiffness of a support apparatus, the method comprising: providing a body adapted to support a body part of a user, providing at least one stiffness adjusting member adapted to be mounted to said body to adjust the stiffness of the apparatus, wherein said body comprises first engaging means and at least one said stiffness adjusting member comprises respective second engaging means for engaging first engaging means to mount at least one stiffness adjusting member to the body, and mounting at least one said stiffness adjusting member to said body by engaging said first engaging means and said second engaging means.

Said first and/or second engaging means may comprise at least one respective protrusion.

Said first and/or second engaging means may comprise at least one respective recess.

Said first and/or second engaging means may comprise a respective plurality of said recesses and/or protrusions or least one said recess and one said protrusion.

The method may further comprise applying tensile or compressive force to said body prior to mounting at least one said stiffness adjusting member to said body.

This provides the advantage of further increasing the adjustability of the apparatus.

The apparatus may be an orthotic and/or prosthetic apparatus.

Preferred embodiments of the present invention will now be described, by way of example only and not in any limitative sense, with reference to the accompanying drawings, in which:

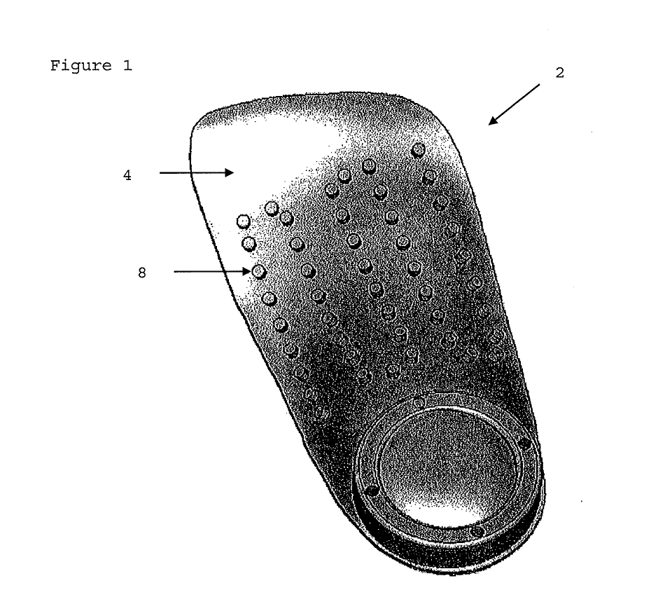

FIG. 1 shows a foot orthotic of a first embodiment of the present invention;

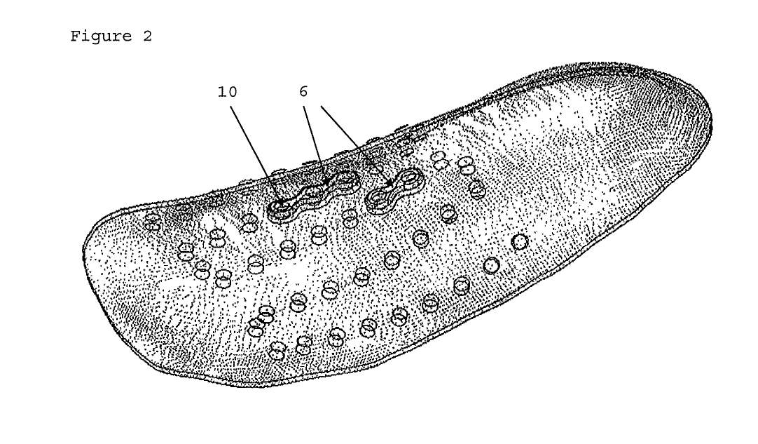

FIG. 2 shows a foot orthotic of a second embodiment of the invention with stiffness adjusting members;

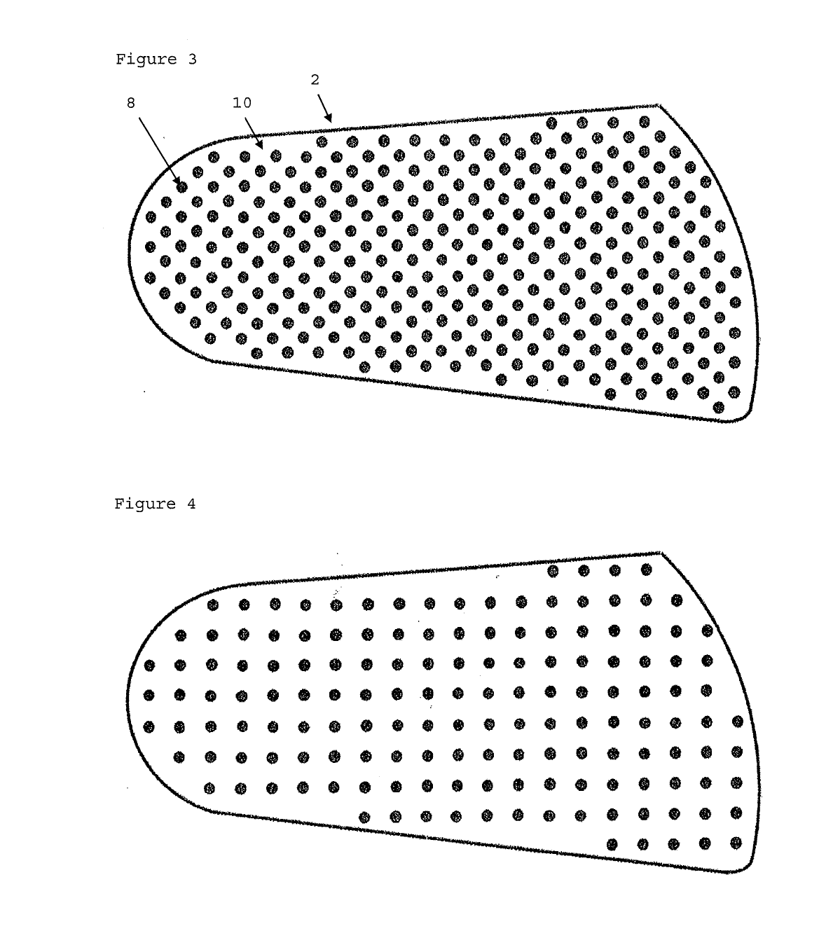

FIGS. 3-7 show a range of possible arrangements of protrusions and/or recesses on the body of the foot orthotic of FIG. 2;

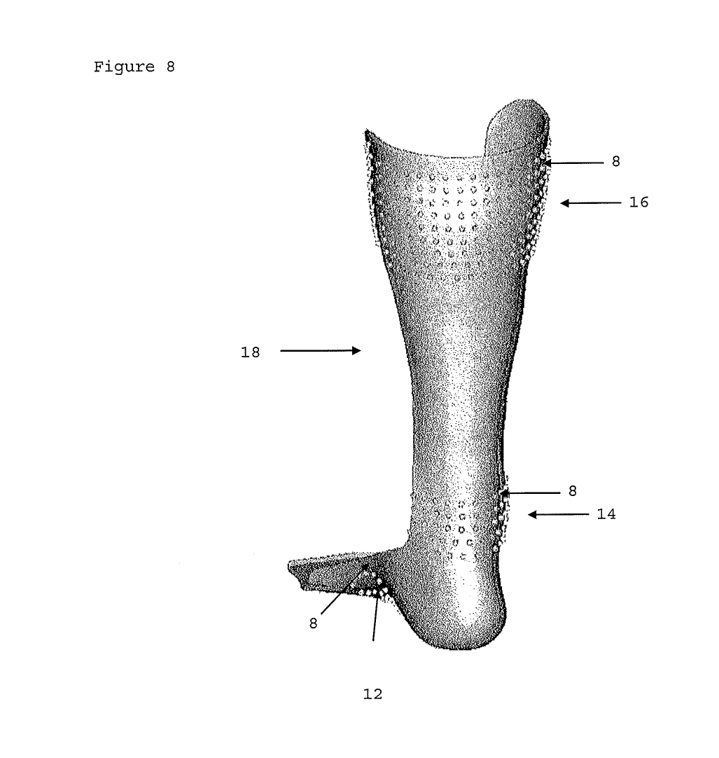

FIG. 8 shows an ankle/foot orthotic of a third embodiment of the invention;

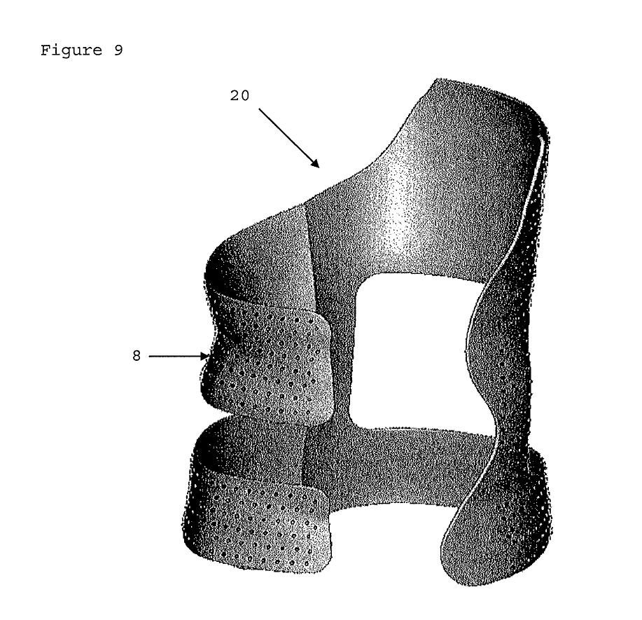

FIG. 9 shows a spinal brace of a fourth embodiment of the invention;

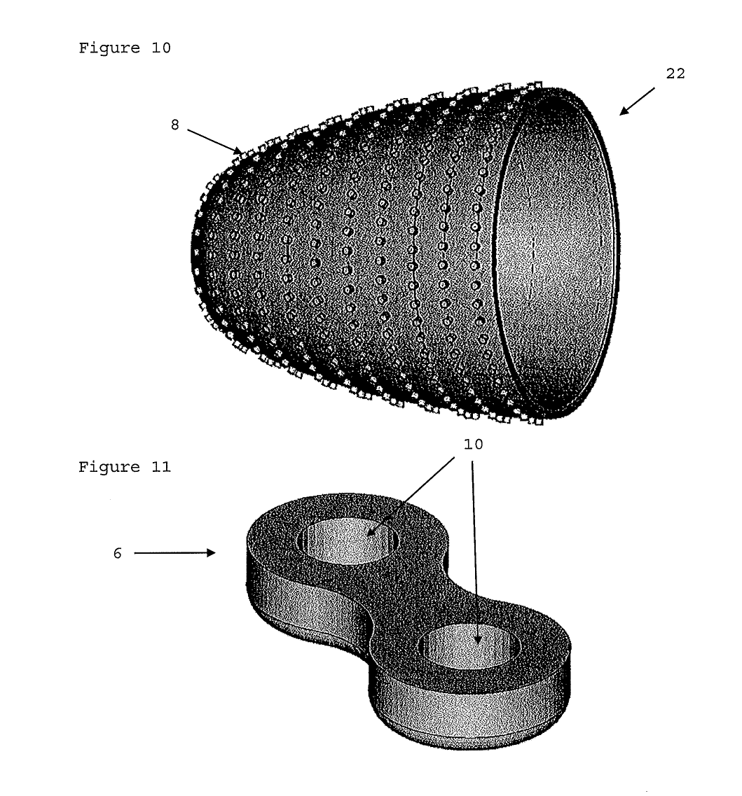

FIG. 10 shows a prosthetic socket of a fifth embodiment of the invention;

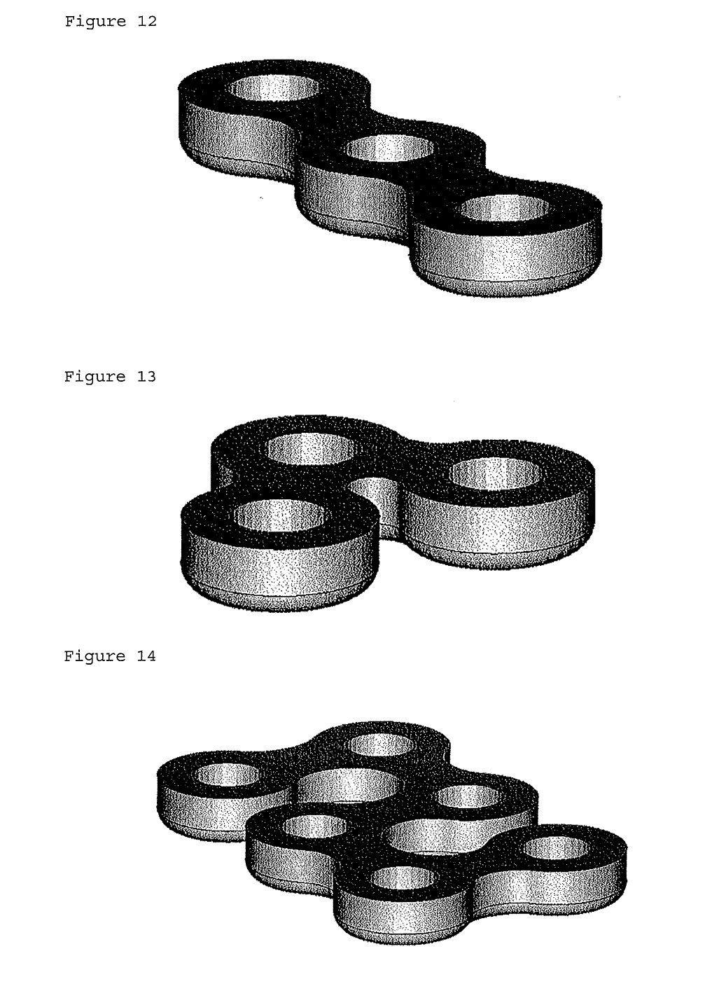

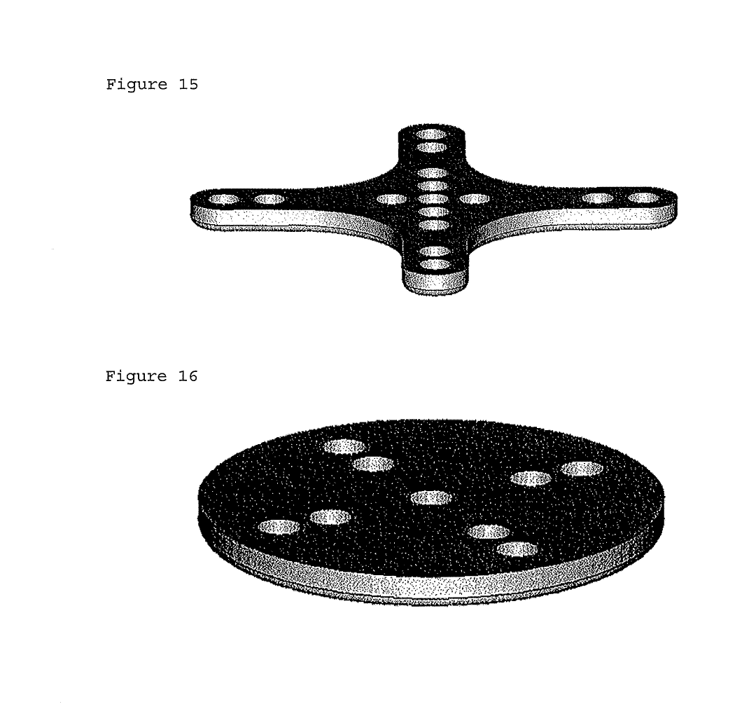

FIGS. 11-16 show a range of possible embodiments of stiffness adjusting members for use with the embodiments of FIGS. 1 to 10;

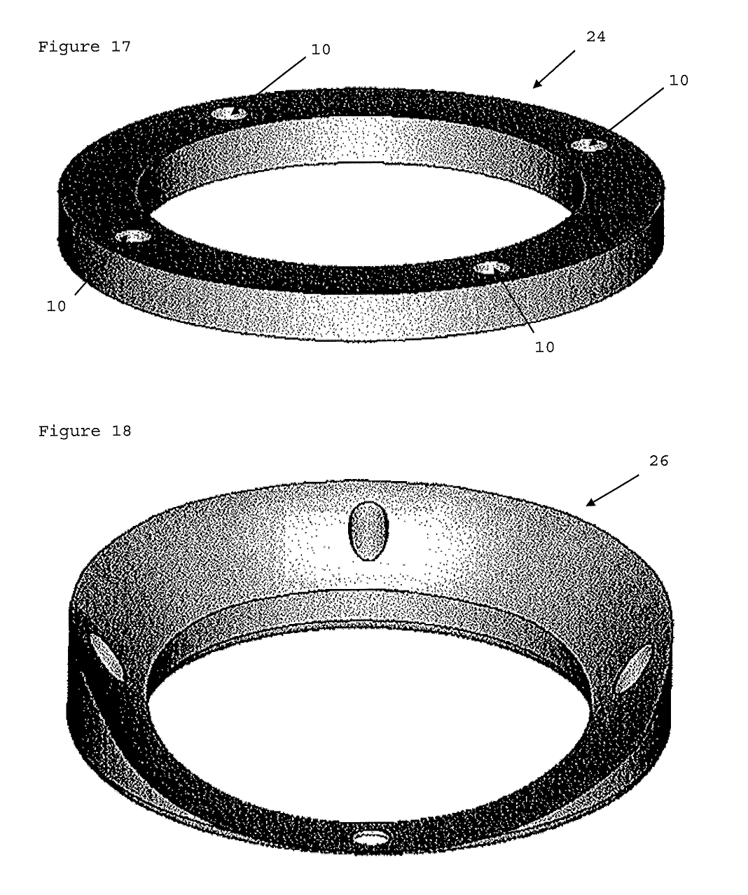

FIG. 17 shows a ring-shaped stiffness adjusting member for use in a foot orthotic apparatus;

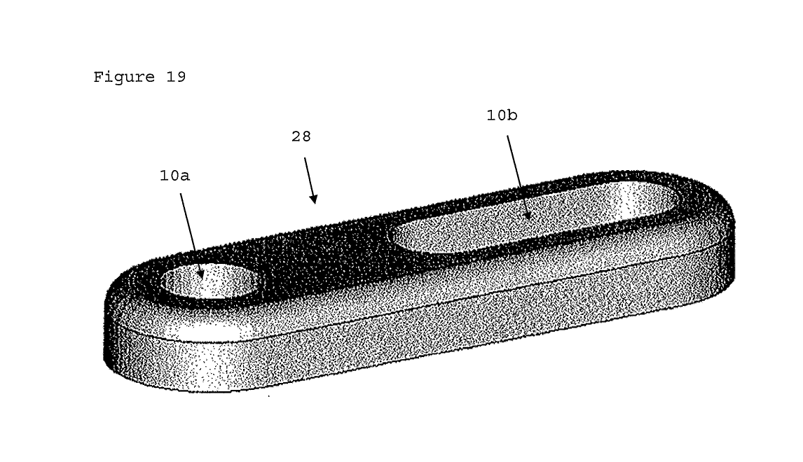

FIG. 18 shows the stiffness adjusting member of FIG. 17 having been contoured; and

FIG. 19 shows a linear stiffness adjusting member.

Orthotic and prosthetic devices are usually manufactured in a very similar way. Initially a plaster impression of the limb or residual limb is taken. This is then filled with plaster or turned into a positive of the limb (i.e. a duplicate). The orientation and/or shape of the limb is then corrected manually to meet a particular clinical requirement. Thermoformable materials are then vacuum formed on the corrected positive to create the body of the orthotic or the prosthetic sockets. Alternatively, a wet lay-up process can be used for composite materials. The device can then be finished with straps, hinges, cushioning pads or other materials and fitted to the patient. Another way of making foot orthoses consists of first designing the orthotic shape via CAD from 3D scanned plaster or foam box impressions and then milling the finished orthotic directly from foam materials, such as EVA foams of different densities. The milled orthotic is then finished manually.

The plaster casting part of the process can be replaced with 3D scanning of the limb (or part of) and designing the device in a CAD environment. Once the correct orthotic or prosthetic socket shape is established, it can be milled from foam materials to create the corrected positive shape and the thermoforming or composite manufacturing process can then be continued. Prosthetic sockets are usually made using thermoplastic materials such as polypropylene or composites, like carbon fibre. For padding orthoses and prosthetic sockets, similar materials are used. These are various kinds of foam such as ethylene-vinyl acetate (EVA) or material supplied under the trade mark Poron (registered trade mark). Also, silicone, leather and different textile materials can be used.

Referring to FIGS. 1 and 2, the underside of a support apparatus in the form of a foot orthotic 2 embodying the present invention is shown having a body 4 for supporting a foot of a wearer and first engaging means in the form of a plurality of cylindrical, stud-like protrusions 8. These protrusions 8 form first engaging means for the two examples of stiffness adjusting member 6 shown in the form of connectors 6 in FIG. 2, whose second engaging means are shown in the form of corresponding recesses 10. It can be seen from FIG. 2 that these examples of stiffness adjusting members 6 are designed to connect together two or more protrusions 8, with the effect of increasing the stiffness of the apparatus 2 in the location of the stiffness adjusting member 6.

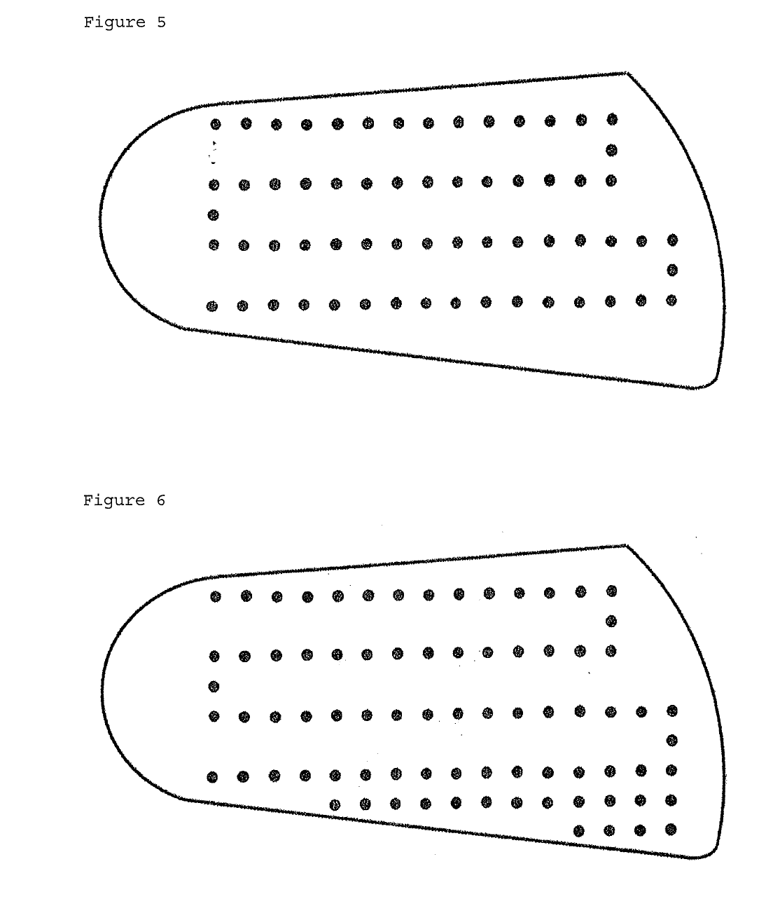

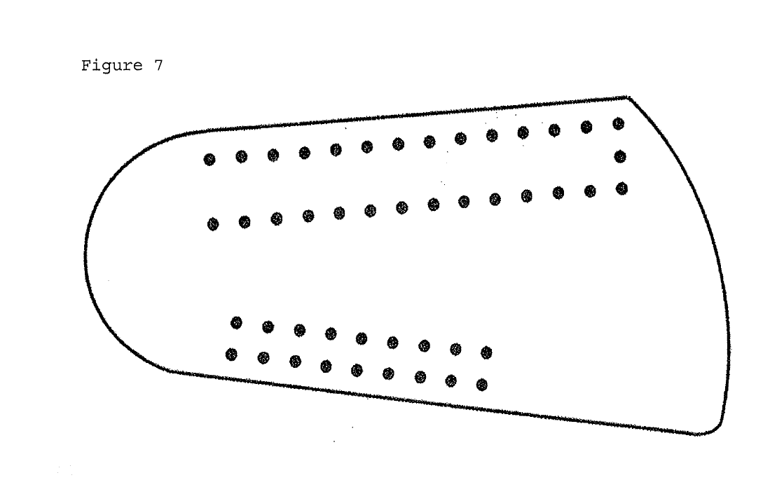

Referring to FIGS. 3-7, a range of ways in which the protrusions 8 can be arranged on the surface of the foot orthotic 2 are shown. FIG. 3 shows a denser arrangement of the protrusions 8, FIGS. 4 and 5 sparser arrangements, and FIGS. 6 and 7 show irregular patterns of protrusions 8. The arrangement of protrusions 8 shown in FIG. 7, for example, demonstrates an emphasis on the requirement to adjust the stiffness of the foot orthotic 2 in the local regions of the arch and outer edge of a user's foot.

Referring to FIG. 8, an ankle/foot orthotic device 18 is shown having an arrangement of protrusions 8 on the outer surface of the device 18 in the regions of the underside of the foot 12, around the ankle region 14, and around the top of the gastrocnemius (calf) muscle region 16.

Referring to FIG. 9, a spinal brace 20 is shown having an arrangement of protrusions 8 on the outer surface of the brace 20.

Referring to FIG. 10, a prosthetic socket 22 is shown having an arrangement of protrusions 8 on the outer surface of the socket 22.

Referring to FIGS. 11 to 16, examples of stiffness adjusting members 6 are shown wherein the second engaging means takes the form of recesses 10 in the stiffness adjusting members 6.

The shape, size, and material of a stiffness adjusting member 6 can all be chosen prior to use in order to adjust the stiffness of one or more regions of an orthotic device 2. For example, a stiffness adjusting member 6 made from metal in the form of the connector 6 shown in FIG. 11 could be chosen to be engaged with protrusions 8 under the arch of a foot orthotic 2, to increase the stiffness in that region to a greater extent than would a connector 6 made from, for example, rubber. In contrast, the same connector 6 may be made from rubber to provide a more yielding adjustment to an orthotic 2 when that adjustment to stiffness is required.

Referring to FIGS. 17 and 18, a stiffness adjusting member 6 is shown in the form of a circular connector 24 with four recesses 10 as second engaging means. A stiffness adjusting member 6, such as the circular connector 24 of FIG. 17, can be contoured like the connector 26 shown in FIG. 18 to provide a better fit to a part of an orthotic device 2, such as the heel area of a foot orthotic 2 in this example.

Referring to FIG. 19, a stiffness adjusting member 6 is shown in the form of a connector 28 containing two recesses (10a, 10b) as second engaging means, wherein a first recess 10a is longer than a second recess 10b along the connection axis of the connector 28, to restrict bending of the part of the orthotic 2 onto which it is engaged to only the direction parallel to the axis of connection. This provides further possibilities for adjustment of the stiffness of the orthotic 2.

It will be appreciated by persons skilled in the art that the above embodiments have been described by way of example only and not in any limitative sense, and that various alterations and modifications are possible without departure from the scope of the invention as defined by the appended claims. For example, the protrusions 8, as well as being cylindrical, can be any suitable shape, such as half-spherical, triangular, rectangular or square etc.

1. A support apparatus comprising:
a body adapted to support a body part of a user; and
at least one stiffness adjusting member adapted to be mounted to said body to adjust the stiffness of the apparatus;
wherein said body comprises at least one first engaging device and at least one said stiffness adjusting member comprises at least one respective second engaging device for engaging said at least one first engaging device to mount the stiffness adjusting member to the body.

2. The apparatus according to claim 1 wherein said at least one first and/or second engaging device comprises at least one respective protrusion.

3. The apparatus according to claim 1 wherein said first at least one and/or second engaging device comprises at least one respective recess.

4. The apparatus according to claim 2 or 3, wherein at least one said first and/or second engaging device comprises a respective plurality of said recess and/or protrusions or at least one said recess and one said protrusion.

5. The apparatus according to claim 1, wherein said body and/or said at least one first engaging device and/or said at least one second engaging device and/or at least one said stiffness adjusting member is manufactured by additive manufacturing.

6. The apparatus according to claim 1, wherein the apparatus is an orthotic and/or prosthetic apparatus.

7. A method of altering the stiffness of a support apparatus, the method comprising:
providing a body adapted to support a boy part of a user;
providing at least one stiffness adjusting member adapated to be mounted to said body to adjust the stiffness of the apparatus;
wherein said body comprises at least one first engaging device and at least one said stiffness adjusting member comprises at least one respective second engaging device for engaging said at least one first engaging device to mount at least one stiffness adjusting member to the body; and
mounting at least one said stiffness adjusting member to said body by engaging said at least one first engaging device and said at least one second engaging device.

8. The method according to claim 7 wherein said at least one first and/or second engaging device comprises at least one respective protrusion.

9. The method according to claim 7 wherein said at least one first and/or second engaging device comprises at least one respective recess.

10. The method according to claim 8, wherein said at least one first and/or second engaging device comprises a respective plurality of said recesses and/or protrusions or at least one said recess and one said protrusion.

11. The method according to claim 7, further comprising:
Applying tensile or compressive force to said body prior to mounting at least one said stiffness adjusting member to said body.

12. The method according to claim 7, wherein the apparatus is an orthotic and/or prosthetic apparatus.

13. The apparatus according to claim 3, wherein at least one said first and/or second engaging device comprises a respective plurality of said recess and/or protrusions or at least one said recess and one said protrusion.

14. The method according to claim 9, wherein said at least one first and/or second engaging device comprises a respective plurality of said recesses and/or protrusions or at least one said recess and one said protrusion.
